(19) United States
(12) Patent Application Publication
(10) Pub. No.: US 2024/0374808 A1
(43) Pub. Date: Nov. 14, 2024

(54) PRESSURE REGULATOR FOR OCULAR SURGERY

(71) Applicant: JOHNSON & JOHNSON SURGICAL VISION, INC., Irvine, CA (US)

(72) Inventors: Alexander Shechtman, Haifa (IL); Assaf Govari, Haifa (IL); Ilya Sitnitsky, Nahariya (IL); Sergey Sobol, Haifa (IL); Alon Boumendil, Givat Nili (IL)

(21) Appl. No.: 18/144,799

(22) Filed: May 8, 2023

(51) Int. Cl. A61M 3/02; A61F 9/007

(52) U.S. Cl. CPC A61M 3/0202; A61F 9/00745; A61B 2217/007; A61M 2205/3344

(57) ABSTRACT

A system includes an ocular surgical handpiece including an irrigation conduit. The system further includes an irrigation tube, configured to establish fluidic communication between a supply of irrigation fluid and the irrigation conduit, such that the irrigation fluid flows distally from the supply, via the irrigation tube and the irrigation conduit, to an eye of a patient. The system further includes a pressure regulator, configured to regulate an intraocular pressure of the eye by regulating the flow of the irrigation fluid within the irrigation conduit, within the irrigation tube, or between the irrigation conduit and the irrigation tube. Other examples are also described.

### FIELD OF THE INVENTION

The present disclosure is related generally to the field of medical devices, and particularly to ocular surgical devices.

### BACKGROUND OF THE INVENTION

During some surgical procedures on an eye, the eye is continuously irrigated with an irrigation fluid, such as a balanced salt solution, while fluid and debris are aspirated from the eye.

### BRIEF DESCRIPTION OF THE DRAWINGS

The present disclosure will be more fully understood from the following detailed description of examples thereof, taken together with the drawings, in which:

FIG. 1 is a schematic illustration of an ocular surgical system, in accordance with some examples of the present disclosure;

FIGS. 2A-B are schematic cross-sectional views of a pressure regulator, in accordance with some examples of the present disclosure;

FIG. 3 is a schematic illustration of an ocular surgical handpiece coupled with a pressure regulator, in accordance with some examples of the present disclosure; and

FIG. 4 is a schematic illustration of an ocular surgical handpiece comprising a pressure regulator, in accordance with some examples of the present disclosure.

### DETAILED DESCRIPTION

### Overview

During an ocular surgical procedure, it is generally desirable to maintain a stable intraocular pressure (IOP). However, for procedures in which fluid is cycled through the eye, it may be challenging to achieve this objective, particularly in view of the volatile nature of the aspiratory flow. For example, if the aspiratory flow suddenly ceases (e.g., due to a blockage), the continued flow of irrigation fluid into the eye may cause the IOP to spike.

To address this challenge, examples of the present disclosure provide a pressure regulator configured to regulate the flow of the irrigation fluid so as to achieve a relatively stable IOP. Advantageously, the pressure regulator is placed downstream (i.e., distally) from the pump that supplies the irrigation fluid, such that the pressure regulator is relatively close to the eye, and hence, responds quickly to changes in IOP. For example, the pressure regulator may be integrated with the handpiece used to perform the surgery. Alternatively, the pressure regulator may be integrated with the tube that carries the irrigation fluid to the handpiece, or placed between the tube and the handpiece.

In some examples, the pressure regulator comprises a chamber configured to contain a gas, such as air, at a predefined threshold pressure. The chamber is separated from the irrigation fluid by a diaphragm, which is coupled with a stopper via a shaft. As the pressure of the irrigation fluid within the pressure regulator, which closely tracks the IOP, rises, the diaphragm moves into the chamber, thereby pulling the stopper into the flow of irrigation fluid such that the stopper at least partly obstructs the flow.

### System Description

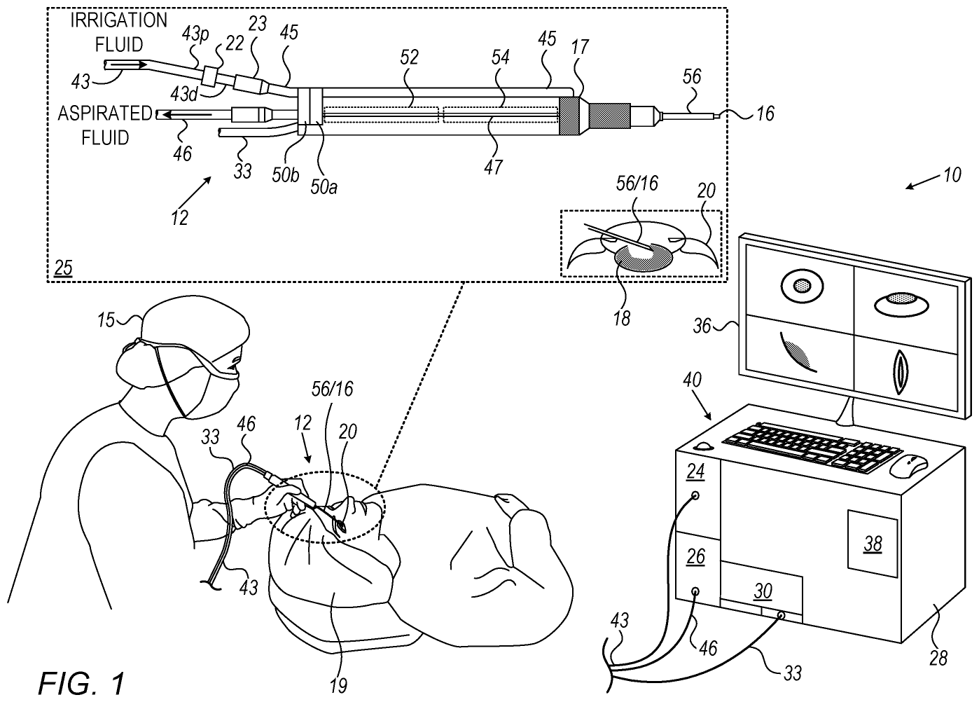

Reference is initially made to FIG. 1, which is a schematic illustration of an ocular surgical system 10, in accordance with some examples of the present disclosure.

System 10 comprises an ocular surgical handpiece 12, which is used by a physician 15 to perform a surgical procedure on an eye 20 of a patient 19. Handpiece 12 comprises an irrigation conduit 45.

System 10 further comprises an irrigation tube 43, which is configured to establish fluidic communication between a supply of irrigation fluid and irrigation conduit 45. The irrigation fluid thus flows distally from the supply, via irrigation tube 43 and irrigation conduit 45, into eye 20.

Typically, irrigation tube 43 is made of a flexible polymer such as polysiloxane or polyvinyl chloride (PVC). Handpiece 12, including irrigation conduit 45, may be made of a polymer or a metal.

The supply of irrigation fluid comprises a reservoir (not shown). Typically, the supply further comprises a pump 24, which pumps the fluid, from the reservoir, into irrigation tube 43. In other examples, the supply of the fluid from the reservoir into irrigation tube 43 is driven by gravity.

In some examples, handpiece 12 is a phacoemulsification handpiece. In such examples, handpiece 12 typically comprises a handpiece body 17, which comprises irrigation conduit 45, and a needle 16 coupled with the distal end of handpiece body 17 and configured to vibrate so as to emulsify a clouded lens 18 of eye 20. Typically, needle 16 is hollow, so as to provide an aspiration channel for fluid, including irrigation fluid, and debris, including emulsified lens material. In particular, handpiece body 17 may be shaped to define an aspiration channel 47, and the fluid and debris may be aspirated through needle 16, via aspiration channel 47, into an aspiration tube 46.

Handpiece 12 may further comprise an ultrasonic actuator 52, which may comprise one or more piezoelectric crystals, and a horn 54 that couples ultrasonic actuator 52 with needle 16. Ultrasonic actuator 52 may thus vibrate needle 16 via horn 54.

Handpiece 12 may further comprise an irrigation sleeve 56 that at least partially surrounds needle 16 and defines a fluid pathway between the external wall of the needle and the internal wall of the irrigation sleeve. Irrigation sleeve 56 may have one or more side ports at, or near, the distal end of the sleeve. The irrigation fluid may thus flow from irrigation conduit 45 into the fluid pathway, and from the distal end of the fluid pathway, and/or the side ports, into the eye.

In some examples, handpiece 12 comprises an anti-vacuum surge (AVS) module 50a comprising, for example, a cartridge configured to couple with a proximal connector 50b. AVS module 50a is configured to facilitate the transfer of energy and/or data to handpiece 12 via magnetic induction. Moreover, AVS module 50a comprises a valve configured to regulate the aspiration through aspiration channel 47, and in particular, to prevent vacuum surges through the aspiration channel. Further details regarding AVS module 50a are provided, for example, in U.S. application Ser. No. 18/116,211, which is assigned to the assignee of the present patent application.

Typically, system 10 further comprises a console 28 containing the supply of irrigation fluid. The proximal end of irrigation tube 43 is configured to couple with console 28 so as to establish fluidic communication with the supply.

Console 28 may further contain an aspiration pump 26, and the proximal end of aspiration tube 46 may be configured to couple with the console so as to establish fluidic communication with pump 26. Pump 26 may thereby pump the fluid and debris from the aspiration tube into a collection receptacle.

Console 28 may further contain an ultrasonic (e.g., module 30 configured to couple with piezoelectric) drive ultrasonic actuator 52 via a cable 33. The console may further contain a controller 38 configured to control drive module 30 in response to instructions received, for example, via a user interface 40.

In some examples, system 10 further comprises a display 36 configured to display images and/or data for facilitating the procedure. Optionally, display 36 and user interface 40 may be combined, in that the display may comprise a touch screen configured to receive input.

As further described below with reference to the subsequent figures, system 10 further comprises a pressure regulator 22, configured to regulate the intraocular pressure of eye 20 by regulating the flow of the irrigation fluid within the irrigation conduit, within the irrigation tube, or between the irrigation conduit and the irrigation tube.

In some examples, as shown in FIG. 1, irrigation tube 43 comprises a proximal segment 43p and a distal segment 43d, and pressure regulator 22 is coupled with the irrigation tube between proximal segment 43p and distal segment 43d such that the pressure regulator regulates the flow within the irrigation tube. Proximal segment 43p comprises the proximal end of the irrigation tube and is thus configured to couple with the supply of irrigation fluid, while distal segment 43d is configured to couple with irrigation conduit 45 (e.g., via a Luer lock 23). Typically, distal segment 43d is relatively short, such that pressure regulator 22 is relatively close to the eye. For example, the length of distal segment 43d may be less than 10 cm.

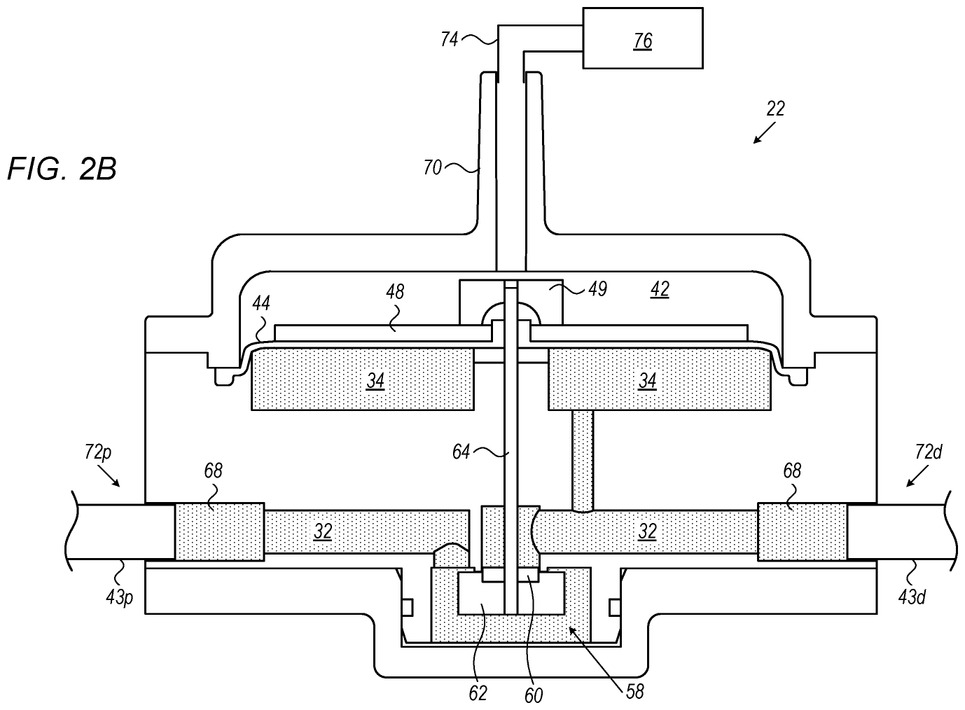

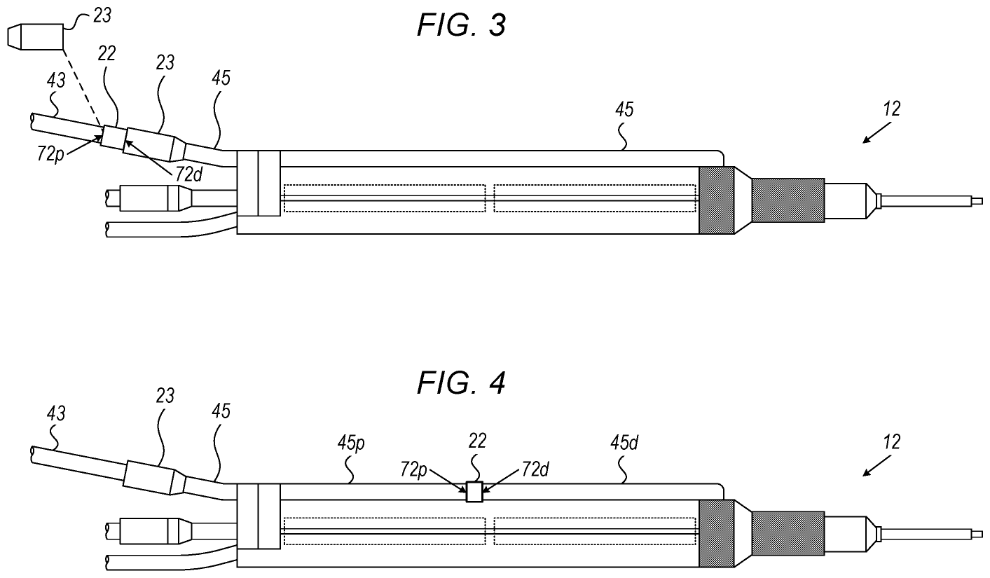

In other examples, the pressure regulator is positioned differently, as described below with reference to FIGS. 3-4.

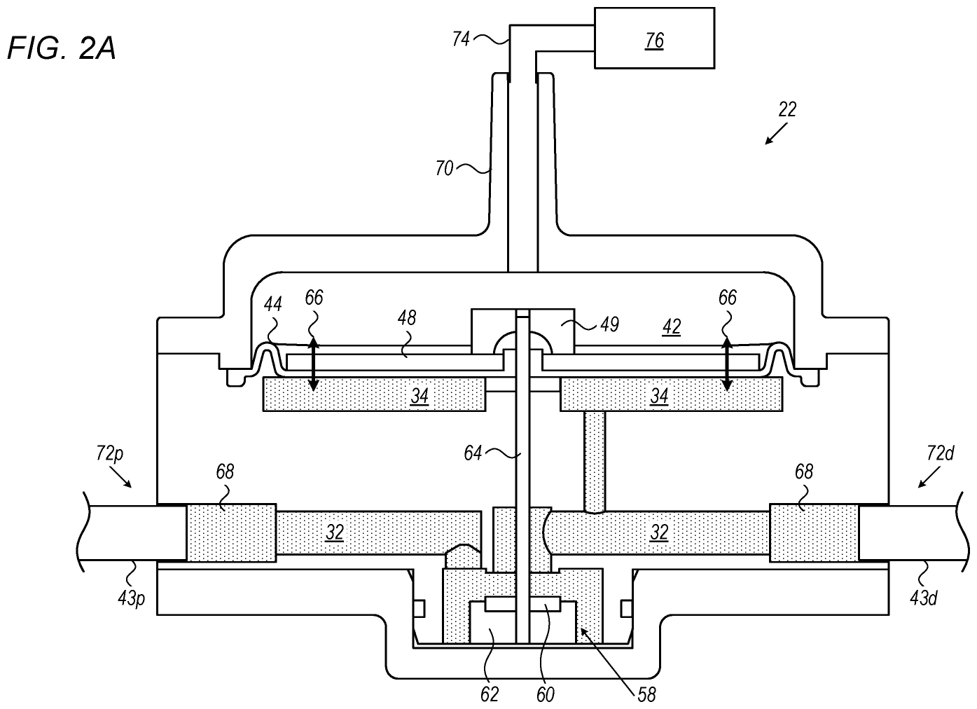

Reference is now made to FIG. 2A, which is a schematic cross-sectional view of pressure regulator 22, in accordance with some examples of the present disclosure. FIG. 2A shows pressure regulator 22 in an open state, in which the pressure regulator does not inhibit the flow of irrigation fluid therethrough. In some examples, pressure regulator 22 is shaped to define a pressure-regulator conduit 32, through which the irrigation fluid flows, and an irrigation-fluid chamber 34 in fluidic communication with pressure-regulator conduit 32. (A hatch pattern indicates portions of the pressure regulator through which the irrigation fluid flows.) The pressure regulator is further shaped to define a control chamber 42, configured to contain a gas (e.g., air) at a predefined threshold pressure. The pressure regulator comprises a diaphragm 44 disposed between irrigation-fluid chamber 34 and control chamber 42 at a position that varies with a pressure differential between the control chamber and the irrigation-fluid chamber. In other words, as the difference between the pressure in irrigation-fluid chamber 34 and the pressure in control chamber 42 changes, diaphragm 44 moves toward or away from the control chamber, as indicated by movement indicators 66 in FIG. 2A. (Optionally, a support 48, which is pressed against diaphragm 44 by a fastener 49, may inhibit the central portion of the diaphragm from flexing, such that the central portion of the diaphragm remains flat as it moves.)

In such examples, pressure regulator 22 further comprises a stopper 58 and a shaft 64 that couples stopper 58 with diaphragm 44, such that, when the pressure within irrigation-fluid chamber 34 exceeds the threshold pressure, the diaphragm pulls the stopper into pressure-regulator conduit 32, thereby slowing (e.g., stopping) the flow of the irrigation fluid. As the flow slows, the pressure in the irrigation-fluid chamber drops. The diaphragm thus moves back toward the irrigation-fluid chamber 34, thereby pushing the valve out of pressure-regulator conduit 32.

As shown in FIG. 2A, stopper 58 may comprise a plug 60, which is configured to enter (and thus obstruct) pressure-regulator conduit 32, and a sealer 62 coupled with plug 60.

Typically, pressure regulator 22 further comprises a gas-supply port 70 in fluidic communication with control chamber 42. Via gas-supply port 70 and an optional tube 74, gas is supplied, from a gas supply 76, to control chamber 42, prior to the procedure, until the pressure within the control chamber reaches the threshold. Optionally, during the procedure (i.e., after activating handpiece 12 (FIG. 1)), the threshold pressure may be adjusted, via gas-supply port 70, by adjusting the amount (i.e., the number of molecules) of the gas within the control chamber 42.

For example, gas supply 76 may comprise a pump, which may pump the gas into the control chamber until the threshold pressure is reached. Subsequently, the pump may be disconnected and gas-supply port 70 may be sealed. Alternatively, the pump may remain connected during the procedure, and, optionally, may pump gas into or out from control chamber 42 so as to adjust the threshold pressure.

Alternatively, for example, gas supply 76 may comprise a reservoir of the gas, in which the gas is at the threshold pressure. Optionally, during the procedure, to change the threshold pressure, control chamber 42 may be coupled with another reservoir at a different pressure. Alternatively, a pressure regulator (not shown) coupled with the reservoir may be used to regulate the pressure within the control chamber during the procedure.

As described above with reference to FIG. 1, pressure regulator 22 may be disposed between proximal segment 43p and distal segment 43d of irrigation tube 43. For example, proximal segment 43p may be coupled with the internal wall 68 of pressure-regulator conduit 32 at a proximal port 72p of the pressure-regulator conduit, and distal segment 43d may be coupled with internal wall 68 at a distal port 72d of the pressure-regulator conduit. This coupling may be effected, for example, via application of an adhesive, injection molding, or welding.

Reference is now made to FIG. 2B, which is another schematic cross-sectional view of pressure regulator 22, in accordance with some examples of the present disclosure. FIG. 2B shows pressure regulator 22 in a closed state, in which plug 60 of stopper 58 has entered pressure-regulator conduit 32, thus inhibiting the flow of irrigation fluid as described above with reference to FIG. 2A.

Notwithstanding FIGS. 2A-B, it is noted that the scope of the present disclosure includes the use of an electronic pressure regulator or any other suitable type of pressure regulator.

Reference is now made to FIG. 3, which is a schematic illustration of ocular surgical handpiece 12 coupled with pressure regulator 22, in accordance with some examples of the present disclosure.

In some examples, pressure regulator 22 is disposed between irrigation tube 43 and irrigation conduit 45, such that the pressure regulator regulates the flow between these two elements. Irrigation tube 43 thus couples with the irrigation conduit via the pressure regulator.

In some such examples, proximal port 72p of pressure regulator 22 is coupled (typically, fixedly) with the distal end of irrigation tube 43. For example, the distal end of the irrigation tube may be coupled with internal wall 68 of proximal port 72p, e.g., via application of an adhesive, injection molding, or welding, as described above, with reference to FIG. 2A, for proximal segment 43p. Distal port 72d of pressure regulator 22 is configured to couple (typically, reversibly) with irrigation conduit 45, e.g., via Luer lock 23. Thus, the distal end of the irrigation tube may be coupled with ocular surgical handpiece 12 by coupling distal port 72d of pressure regulator 22 with irrigation conduit 45.

In other such examples, distal port 72d of pressure regulator 22 is coupled (typically, fixedly) with irrigation conduit 45. For example, the proximal end of irrigation conduit 45 may be coupled with internal wall 68 of distal port 72d, e.g., via application of an adhesive, injection molding, or welding, as described above, with reference to FIG. 2A, for distal segment 43d. Proximal port 72p of pressure regulator 22 is configured to couple (typically, reversibly) with the distal end of irrigation tube 43, e.g., via Luer lock 23. Thus, the distal end of irrigation tube 43 may be coupled with ocular surgical handpiece 12 by coupling the distal end of irrigation tube 43 with proximal port 72p of pressure regulator 22.

In yet other such examples, proximal port 72p of pressure regulator 22 is configured to couple (typically, reversibly) with the distal end of irrigation tube 43 (e.g., via a first Luer lock 23), and the distal port of the pressure regulator is configured to couple (typically, reversibly) with irrigation conduit 45 (e.g., via a second Luer lock 23).

Reference is now made to FIG. 4, which is a schematic illustration of ocular surgical handpiece 12 comprising pressure regulator 22, in accordance with some examples of the present disclosure.

In some examples, irrigation conduit 45 comprises a proximal segment 45p, configured to couple with irrigation tube 43 (e.g., via Luer lock 23), and a distal segment 45d. Pressure regulator 22 is coupled with irrigation conduit 45 between proximal segment 45p and distal segment 45d, e.g., as described above, with reference to FIG. 2A, for proximal segment 43p and distal segment 43d. Pressure regulator 22 thus regulates the flow within irrigation conduit 45.

### EXAMPLES

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes.

It is contemplated that the various teachings herein may be arranged d applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A system (10) includes an ocular surgical handpiece (12) including an irrigation conduit (45). The system further includes an irrigation tube (43), configured to establish fluidic communication between a supply of irrigation fluid and the irrigation conduit, such that the irrigation fluid flows distally from the supply, via the irrigation tube and the irrigation conduit, to an eye (20) of a patient (19). The system further includes a pressure regulator (22), configured to regulate an intraocular pressure of the eye by regulating the flow of the irrigation fluid within the irrigation conduit, within the irrigation tube, or between the irrigation conduit and the irrigation tube.

### Example 2

The system (10) according to Example 1,
wherein the irrigation tube (43) includes:
   a proximal segment (43p); and
   a distal segment (43d), configured to couple with the irrigation conduit (45), and
wherein the pressure regulator (22) is coupled with the irrigation tube between the proximal segment and the distal segment such that the pressure regulator regulates the flow within the irrigation tube.

### Example 3

The system (10) according to Example 2, wherein a length of the distal segment (43d) is less than 10 cm.

### Example 4

The system (10) according to Example 1, wherein the pressure regulator (22) includes:
a proximal port (72p) coupled with a distal end of the irrigation tube (43); and
a distal port (72d) configured to couple with the irrigation conduit (45) such that the pressure regulator regulates the flow between the irrigation conduit and the irrigation tube.

### Example 5

The system (10) according to Example 1, wherein the pressure regulator (22) includes:
a distal port (72d) coupled with the irrigation conduit (45); and
a proximal port (72p) configured to couple with a distal end of the irrigation tube (43) such that the pressure regulator regulates the flow between the irrigation conduit and the irrigation tube.

### Example 6

The system (10) according to Example 1, wherein the pressure regulator (22) includes:
a proximal port (72p) configured to couple with a distal end of the irrigation tube (43); and
a distal port (72d) configured to couple with the irrigation conduit (45) such that the pressure regulator regulates the flow between the irrigation conduit and the irrigation tube.

### Example 7

The system (10) according to Example 1,
wherein the irrigation conduit (45) includes:
   a proximal segment (45p), configured to couple with the irrigation tube (43); and
   a distal segment (45d), and
wherein the pressure regulator (22) is coupled with the irrigation conduit between the proximal segment and the distal segment such that the pressure regulator regulates the flow within the irrigation conduit.

### Example 8

The system (10) according to any one of Examples 1-7, wherein the ocular surgical handpiece (12) is a phacoemulsification handpiece.

### Example 9

The system (10) according to any one of Examples 1-8,
wherein the pressure regulator (22) is shaped to define:
   a pressure-regulator conduit (32), through which the irrigation fluid flows,
   an irrigation-fluid chamber (34) in fluidic communication with the pressure-regulator conduit, and
   a control chamber (42), configured to contain a gas at a predefined threshold pressure, and
wherein the pressure regulator includes:
   a diaphragm (44) disposed between the irrigation-fluid chamber and the control chamber at a position that varies with a pressure differential between the control chamber and the irrigation-fluid chamber;
   a stopper (58); and
   a shaft (64) that couples the stopper with the diaphragm such that, when a pressure within the irrigation-fluid chamber exceeds the threshold pressure, the diaphragm pulls the stopper into the pressure-regulator conduit, thereby slowing the flow of the irrigation fluid.

### Example 10

The system (10) according to Example 9, wherein the pressure regulator (22) further includes a gas-supply port (20) in fluidic communication with the control chamber (42) and configured for a supply of the gas, via the gas-supply port, to the control chamber.

### Example 11

A method for controlling intraocular pressure includes providing a surgical system (10) including an ocular surgical handpiece (12) having an irrigation conduit (45) and an aspiration channel (47), an irrigation tube (43) and an aspiration tube (46), wherein the irrigation tube and the aspiration tube are each coupled with a proximal end of the ocular surgical handpiece and coupled with the irrigation conduit and aspiration channel, respectively, and at least one pump (26) coupled with the ocular surgical handpiece via the aspiration tube and configured to aspirate irrigation fluid and lens material from an eye (20). The method further includes coupling a proximal end of the irrigation tube with a supply of the irrigation fluid, thereby establishing fluidic communication between the supply and the irrigation conduit such that the irrigation fluid flows distally from the supply to the eye, via the irrigation tube. The method further includes coupling a pressure regulator (22) with the irrigation conduit or the irrigation tube. The pressure regulator includes a pressure-regulator conduit (32), through which the irrigation fluid flows, an irrigation-fluid chamber (34) in fluidic communication with the pressure-regulator conduit, and a control chamber (42), configured to contain a gas at a predefined threshold pressure. The pressure regulator includes a diaphragm (44) disposed between the irrigation-fluid chamber and the control chamber at a position that varies with a pressure differential between the control chamber and the irrigation-fluid chamber, a stopper (58), and a shaft (64) that couples the stopper with the diaphragm. The method further includes supplying the irrigation fluid to the eye, activating the ocular surgical handpiece to emulsify a lens (18) of the eye, activating the pump to aspirate the emulsified lens and irrigation fluid, and providing the threshold pressure to the pressure regulator. When a pressure within the irrigation-fluid chamber exceeds the threshold pressure, the diaphragm pulls the stopper into the pressure-regulator conduit via the shaft, thereby slowing the flow of the irrigation fluid and regulating the intraocular pressure of the eye by regulating a flow of the irrigation fluid within the irrigation conduit, within the irrigation tube, or between the irrigation conduit and the irrigation tube.

### Example 12

The method according to Example 11,
wherein the irrigation tube (43) includes:
   a proximal segment (43p), including the proximal end of the irrigation tube, and
   a distal segment (43d), which is coupled with the proximal end of the ocular surgical handpiece (12), and
wherein coupling the pressure regulator (22) with the irrigation tube includes coupling the pressure regulator with the irrigation tube between the proximal segment and the distal segment.

### Example 13

The method according to Example 12, wherein a length of the distal segment (43d) is less than 10 cm.

### Example 14

The method according to Example 11, wherein coupling the pressure regulator (22) with the irrigation conduit (45) or the irrigation tube (43) includes coupling the pressure regulator between the irrigation conduit and the irrigation tube.

### Example 15

The method according to any one of Examples 11-14, further including, subsequently to activating the ocular surgical handpiece (12), adjusting the threshold pressure by adjusting an amount of the gas within the control chamber (42).

It will be appreciated by persons skilled in the art that the present disclosure is not limited to what has been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system, comprising:
an ocular surgical handpiece comprising an irrigation conduit;
an irrigation tube, configured to establish fluidic communication between a supply of irrigation fluid and the irrigation conduit, such that the irrigation fluid flows distally from the supply, via the irrigation tube and the irrigation conduit, to an eye of a patient; and
a pressure regulator, configured to regulate an intraocular pressure of the eye by regulating the flow of the irrigation fluid within the irrigation conduit, within the irrigation tube, or between the irrigation conduit and the irrigation tube.

2. The system according to claim 1,
wherein the irrigation tube comprises:
a proximal segment; and
a distal segment, configured to couple with the irrigation conduit, and
wherein the pressure regulator is coupled with the irrigation tube between the proximal segment and the distal segment such that the pressure regulator regulates the flow within the irrigation tube.

3. The system according to claim 2, wherein a length of the distal segment is less than 10 cm.

4. The system according to claim 1, wherein the pressure regulator comprises:
a proximal port coupled with a distal end of the irrigation tube; and
a distal port configured to couple with the irrigation conduit such that the pressure regulator regulates the flow between the irrigation conduit and the irrigation tube.

5. The system according to claim 1, wherein the pressure regulator comprises:
a distal port coupled with the irrigation conduit; and
a proximal port configured to couple with a distal end of the irrigation tube such that the pressure regulator regulates the flow between the irrigation conduit and the irrigation tube.

6. The system according to claim 1, wherein the pressure regulator comprises:
a proximal port configured to couple with a distal end of the irrigation tube; and
a distal port configured to couple with the irrigation conduit such that the pressure regulator regulates the flow between the irrigation conduit and the irrigation tube.

7. The system according to claim 1,
wherein the irrigation conduit comprises:
a proximal segment, configured to couple with the irrigation tube; and
a distal segment, and
wherein the pressure regulator is coupled with the irrigation conduit between the proximal segment and the distal segment such that the pressure regulator regulates the flow within the irrigation conduit.

8. The system according to claim 1, wherein the ocular surgical handpiece is a phacoemulsification handpiece.

9. The system according to claim 1,
wherein the pressure regulator is shaped to define:
a pressure-regulator conduit, through which the irrigation fluid flows,
an irrigation-fluid chamber in fluidic communication with the pressure-regulator conduit, and
a control chamber, configured to contain a gas at a predefined threshold pressure, and wherein the pressure regulator comprises:
a diaphragm disposed between the irrigation-fluid chamber and the control chamber at a position that varies with a pressure differential between the control chamber and
the irrigation-fluid chamber;
a stopper; and
a shaft that couples the stopper with the diaphragm such that, when a pressure within the irrigation-fluid chamber exceeds the threshold pressure, the diaphragm pulls the stopper into the pressure-regulator conduit, thereby slowing the flow of the irrigation fluid.

10. The system according to claim 9, wherein the pressure regulator further comprises a gas-supply port in fluidic communication with the control chamber and configured for a supply of the gas, via the gas-supply port, to the control chamber.

11. A method for controlling intraocular pressure, the method comprising:
providing a surgical system comprising:
an ocular surgical handpiece having an irrigation conduit and an aspiration channel,
an irrigation tube and an aspiration tube, wherein the irrigation tube and the aspiration tube are each coupled with a proximal end of the ocular surgical handpiece and coupled with the irrigation conduit and aspiration channel, respectively, and
at least one pump coupled with the ocular surgical handpiece via the aspiration tube and configured to aspirate irrigation fluid and lens material from an eye;
coupling a proximal end of the irrigation tube with a supply of the irrigation fluid, thereby establishing fluidic communication between the supply and the irrigation conduit such that the irrigation fluid flows distally from the supply to the eye, via the irrigation tube;
coupling a pressure regulator with the irrigation conduit or the irrigation tube,
wherein the pressure regulator comprises a pressure-regulator conduit, through which the irrigation fluid flows, an irrigation-fluid chamber in fluidic communication with the pressure-regulator conduit, and a control chamber, configured to contain a gas at a predefined threshold pressure, and
wherein the pressure regulator comprises a diaphragm disposed between the irrigation-fluid chamber and the control chamber at a position that varies with a pressure differential between the control chamber and the irrigation-fluid chamber, a stopper, and a shaft that couples the stopper with the diaphragm;
supplying the irrigation fluid to the eye;
activating the ocular surgical handpiece to emulsify a lens of the eye;
activating the pump to aspirate the emulsified lens and irrigation fluid; and
providing the threshold pressure to the pressure regulator,
wherein, when a pressure within the irrigation-fluid chamber exceeds the threshold pressure, the diaphragm pulls the stopper into the pressure-regulator conduit via the shaft, thereby slowing the flow of the irrigation fluid and regulating the intraocular pressure of the eye by regulating a flow of the irrigation fluid within the irrigation conduit, within the irrigation tube, or between the irrigation conduit and the irrigation tube.

12. The method according to claim 11,
wherein the irrigation tube includes:
a proximal segment, including the proximal end of the irrigation tube, and
a distal segment, which is coupled with the proximal end of the ocular surgical handpiece, and
wherein coupling the pressure regulator with the irrigation tube comprises coupling the pressure regulator with the irrigation tube between the proximal segment and the distal segment.

13. The method according to claim 12, wherein a length of the distal segment is less than 10 cm.

14. The method according to claim 11, wherein coupling the pressure regulator with the irrigation conduit or the irrigation tube comprises coupling the pressure regulator between the irrigation conduit and the irrigation tube.

15. The method according to claim 11, further comprising, subsequently to activating the ocular surgical handpiece, adjusting the threshold pressure by adjusting an amount of the gas within the control chamber.
